(19) United States
(12) Patent Application Publication
(10) Pub. No.: US 2016/0089170 A1
(43) Pub. Date: Mar. 31, 2016

(54) INVERTABLE MEDICAL RETRIEVAL DEVICES AND METHODS

(71) Applicant: Boston Scientific Scimed, Inc., Maple Grove, MN (US)

(72) Inventors: Scott A. DAVIS, Southborough, MA (US); Christopher A. BENNING, Hopkinton, MA (US)

(73) Assignee: Boston Scientific Scimed, Inc.

(21) Appl. No.: 14/855,882

(22) Filed: Sep. 16, 2015

Related U.S. Application Data
(60) Provisional application No. 62/057,773, filed on Sep. 30, 2014.

(51) Int. Cl. A61B 17/221

(52) U.S. Cl. CPC A61B 17/221

(57) ABSTRACT

A medical retrieval device may include an elongate sheath, a first and a second control wire extending through the sheath, and a basket coupled to a distal end of the first control wire. The retrieval device may additionally include an engaging member coupled to a distal end of the second control wire and the basket. The engaging member may be configured to transform from a constrained configuration within the sheath to an unconstrained configuration outside to the sheath, wherein transforming the engaging member from the unconstrained configuration to the constrained configuration may rotate the basket about an axis perpendicular to a longitudinal axis of the sheath.

### CROSS-REFERENCE TO RELATED APPLICATIONS

The application claims the benefits of priority from U.S. Provisional Application No. 62/057,773, filed on Sep. 30, 2014, which is incorporated by reference herein in its entirety.

### TECHNICAL FIELD

The disclosure generally relates to medical retrieval devices such as, for example, baskets, for retrieving material from a body of a patient. More particularly, the disclosure generally relates to medical retrieval baskets that may be inverted within the body, and methods of use.

### BACKGROUND

Medical retrieval devices are often utilized for removing organic material (e.g., blood clots, tissue, and biological concretions such as urinary, biliary, and pancreatic stones) and/or inorganic material (e.g., components of a medical device or other foreign matter), which may obstruct or otherwise be present within a patient's body cavities or lumens. For example, concretions can develop in certain parts of the body, such as in the kidneys, pancreas, ureter, and gallbladder. Minimally invasive medical procedures are sometimes used to remove these concretions using medical retrieval baskets inserted into the body through natural orifices, or through an incision.

In some cases, the stone may be too large to be removed intact from the body tract after it has been captured within the basket. In such cases, the user (doctors, medical professionals, etc.) may attempt to dislodge the stone (or other material) from the basket. If the stone cannot be removed from the basket in this manner, the user may try to break, or otherwise fragment, the stone in the basket. However, in some cases, parts of the basket may break and may be released in the body. In such cases, surgery may be required to remove the broken pieces.

### SUMMARY

Embodiments of the present disclosure relate to, among other things, medical retrieval devices and related methods of use. Each of the embodiments disclosed herein may include one or more of the features described in connection with any of the other disclosed embodiments.

In one embodiment, a medical retrieval device is disclosed. The retrieval device may include an elongate sheath, a first and a second control wire extending through the sheath, and a basket coupled to a distal end of the first control wire. The retrieval device may additionally include an engaging member coupled to a distal end of the second control wire and the basket. The engaging member may be configured to transform from a constrained configuration within the sheath to an unconstrained configuration outside to the sheath, wherein transforming the engaging member from the unconstrained configuration to the constrained configuration may rotate the basket about an axis perpendicular to a longitudinal axis of the sheath.

Additionally or alternatively, embodiments of the retrieval device may include one or more of the following features: the basket may be configured to transform from a compressed configuration within the sheath to an expanded configuration outside the sheath; the basket may extend from a proximal end to a distal end, wherein a first end of the engaging member is connected to the proximal end of the basket and an opposite second end of the engaging member is connected to the distal end of the second control wire; in the unconstrained configuration, the engaging member may be substantially U-shaped, and in the constrained configuration, the engaging member may have an elongated shape; the retrieval device may include an elastic wire connecting a distal end of the first control wire and the basket, wherein the elastic wire is more elastic than the first control wire; the elastic wire may be a coiled wire.

Additionally or alternatively, embodiments of the retrieval device may include one or more of the following features: pulling the engaging member into the sheath may transform the engaging member to the constrained configuration and rotate the basket to an inverted configuration; the basket may have a proximal portion and a distal portion, wherein, in the inverted configuration, the proximal portion may be positioned distal to the distal portion; the basket may include a shape-memory material; the engaging member may include a shape-memory material or a spring material; the basket may be configured to extend from within the sheath to outside the sheath, wherein extending the basket from within the sheath to outside the sheath may rotate the basket about the axis; the basket may include a plurality of legs joined together at opposite ends; the basket may include a proximal portion having a first plurality of legs and a distal portion having a second plurality of legs, wherein a gap between adjacent legs of the first plurality of legs may be greater than a gap between adjacent legs of the second plurality of legs; the basket may include an atraumatic tip; the engaging member is a flat wire.

In another embodiment, a medical retrieval device is disclosed. The retrieval device may include a sheath having a lumen, and a basket having a proximal end and a distal end. The basket may be configured to transition between a first configured within the lumen to a second configuration outside the lumen. Wherein in the second configuration, the proximal end of the basket may be positioned proximal to the distal end, and in the first configuration, the proximal end of the basket may be positioned distal to the distal end. The retrieval device may also include an engaging member coupled to the basket and configured to rotate the basket about an axis perpendicular to a longitudinal axis of the sheath as the basket emerges from within the lumen.

Additionally or alternatively, embodiments of the retrieval device may include one or more of the following features: a first control wire and a second control wire extending through the sheath, wherein, a distal end of the first control wire may be coupled to the proximal end of the basket, and a distal end of the second control wire may be connected to a second end of the engaging member; a first end of the engaging member opposite the second end may be connected to the proximal end of the basket; an elastic wire connecting the distal end of the first control wire to the proximal end of the basket; and retracting the engaging member from outside the lumen into the lumen may be configured to rotate the basket about the axis.

In another embodiment, a method of using a medical retrieval device to manipulate an object within a body of a patient is disclosed. The method may include directing a distal end of an elongate hollow sheath into the body, the sheath may including a basket in a first configuration loaded therein. Wherein in the first configuration, a proximal end of the basket may be positioned distal to a distal end of the basket. The method may also include extending the basket from within the sheath to rotate the basket from the first configuration to a second configuration outside the sheath, wherein in the second configuration, the proximal end of the basket is positioned proximal to the distal end. The method may also include capturing the object within the basket.

Additionally or alternatively, the method may include one or more of the following features: the medical retrieval device may include an engaging member coupled to the basket, the engaging member may be configured to transform from an elongated shape within the sheath to a curved shape outside the sheath, and wherein extending the basket includes extending the engaging member out of the sheath; the method may further include retracting the engaging member into the sheath to rotate the basket about an axis perpendicular to a longitudinal axis of the sheath after capturing the object within the basket; the method may also include releasing the object from within the basket; the method may also include inverting the basket after capturing the object to release the object from the basket; and extending the basket from within the sheath may transform the basket from a compressed configuration within the sheath to an expanded configuration outside the sheath.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 illustrates an exemplary medical device of the current disclosure;

FIG. 2 illustrates an exemplary basket of the medical device of FIG. 1;

FIG. 3 illustrates the basket of FIG. 2 rotating to an inverted configuration;

FIG. 4 illustrates the basket of FIG. 2 in an inverted configuration;

FIG. 5 illustrates the basket of FIG. 2 loaded on a sheath and positioned in a body lumen of a patient;

FIG. 6 illustrates the basket of FIG. 2 emerging from the sheath;

FIG. 7 illustrates the basket of FIG. 2 with a stone captured therein; and

FIG. 8 illustrates the basket of FIG. 2 in an inverted configuration releasing the stone in the body lumen.

### DETAILED DESCRIPTION

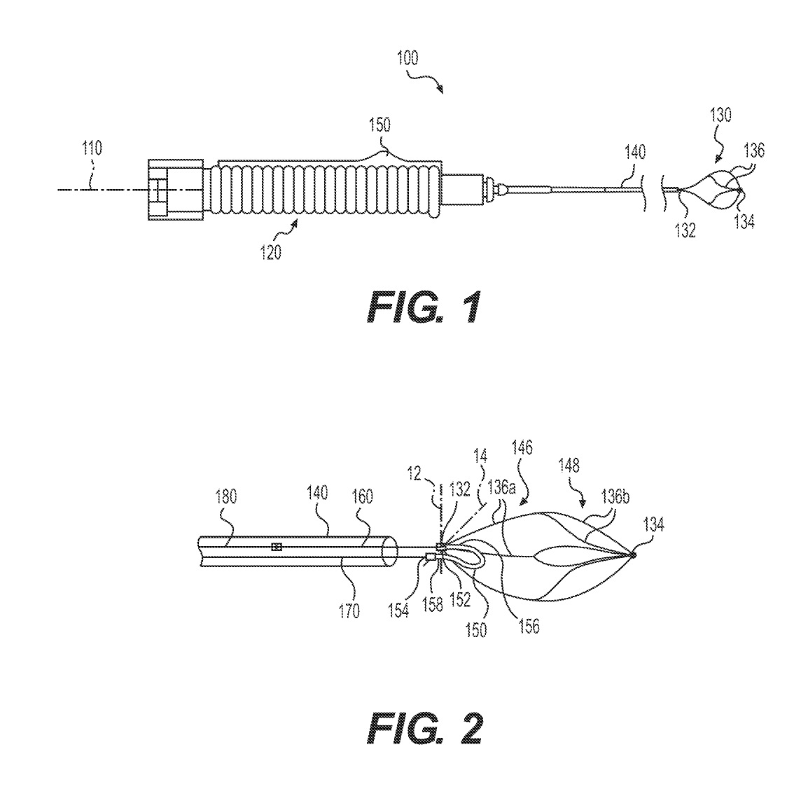

FIG. 1 illustrates an exemplary medical device 100 of the present disclosure. Device 100 includes a handle 120 extending along a central longitudinal axis 110. Handle 120 may be configured to selectively extend and retract a retrieval device (e.g., a basket 130) relative to a sheath 140, as will be described in further detail below. Accordingly, handle 120 may include one or more actuators configured to actuate the basket 130. For example, an actuator 150 may move the basket 130 between a collapsed position within sheath 140 and an expanded position distal to the sheath 140, as will be described in further detail below. Actuator 150 may include, for example, a sliding mechanism, a rotating mechanism, a pushing mechanism, or the like. Although only one actuator 150 is illustrated in FIG. 1, in some embodiments, device 100 may include multiple actuators to actuate the basket 130.

Sheath 140 may include an elongate tube that extends from the handle 120 to a distal end that may be positionable within the body of the patient. A lumen may extend along the length of the sheath 140. In some embodiments, sheath 140 may include a plurality of lumens extending therethrough. Sheath 140 may include any appropriate polyimide tube or a hollow member of another material that exhibits suitable radial flexibility, axial stiffness, and biocompatibility. In general, sheath 140 may have any outer dimension (e.g., diameter) and cross-sectional shape configured for insertion into a body lumen directly or through a channel of an insertion device such as, for example, a scope device (such as a ureteroscope, endoscope, colonoscope, etc.), a catheter, or the like. The sheath 140 may have an inner dimension and cross-sectional shape suitable for the insertion of a basket 130 therethrough.

Basket 130 may include a plurality of legs 136 joined together to form a cage that extends from a proximal end 132 to a distal end 134. In general, basket 130 may have any number or pattern of legs 136. The number and/or pattern of the legs 136 may be selected on the basis of a number of parameters such as, for example, the size of the body lumen/cavity into which the basket 130 is inserted, the size of the stone or other material to be removed, the consistency of the stone or other material, the position of the stone or other material, and the location and/or orientation of the stone or other material within a patient.

FIG. 2 illustrates an exemplary embodiment of basket 130. Basket 130 may include a proximal portion 146 and a distal portion 148. The proximal portion 146 may include a plurality of longitudinally extending proximal legs 136a coupled together at the proximal end 132. The gap between the proximal legs 136a may define an entrance zone for the basket 130 at the proximal portion 146. That is, the basket 130 may be configured to receive a stone (or other object) therein through the gap between the proximal legs 136a. In use, as will be described in further detail below, a user may manipulate (for example, move) the basket 130 such that the stone enters the basket 130 through the entrance zone in the proximal portion 146.

The distal portion 148 of the basket 130 may include a plurality of distal legs 136b extending distally of the proximal legs 136a. In some embodiments, as illustrated in FIG. 2, multiple distal legs 136b may extend distally from each proximal leg 136a. The plurality of distal legs 136b may be coupled together at the distal end 134 of the basket 130 to form a net at the distal portion 148 of the basket 130. The spacing between the distal legs 136b may be configured to prevent the release of a stone therethrough. Once a stone is received in the basket 130 (e.g., through the entrance zone), it may be trapped therein by the net formed by the plurality of distal legs 136b.

The distal legs 136b may be coupled together at the distal end 134, and the proximal legs 136a may be coupled together at the proximal end 132, by any appropriate method including, for example, welding, adhesives, and/or tying. In some embodiments, collars or crimped hypo-tubes at the proximal and/or distal ends 132, 134 may join the respective legs together. In some embodiments, the distal end 134 of the basket 130 may form a tip. In some embodiments, the tip may be rounded to form an atraumatic surface. An atraumatic tip may prevent inadvertent damage to tissue during movement of the basket 130 within a body lumen. In some embodiments, the tip may include a cap with a rounded or otherwise atraumatic end. In some embodiments, the basket 130 may not include a tip. Instead, each distal leg 136b may extend from the distal end of a proximal leg 136a to the distal end of another proximal leg 136a to form a loop.

It should be noted that the pattern of basket 130 illustrated in FIG. 2 is only exemplary. In general, any known pattern and type of basket (three wire, four wire, helical, etc.) may be used as basket 130. In some embodiments, basket 130 may be self-expandable, and may be made at least partially of a shape-memory material. As known in the art, a shape-memory material is a material that “remembers” its original shape. The shape-memory material may be deformed to a deformed shape (or stressed shape) by the application of an external force. When the external forces are released, the material reverts substantially back to its original, stress-free shape. Examples of shape-memory materials include synthetic plastics, stainless steel, and superelastic metallic alloys of nickel/titanium (e.g., Nitinol), copper, cobalt, vanadium, chromium, iron, or the like. Alternative basket materials include, but are not limited to, other metal alloys, powdered metals, ceramics, thermal plastic composites, ceramic composites, and polymers. Also, combinations of these and other materials can be used.

The basket 130 may be coupled to the handle 120 (see FIG. 1) via a first control wire 180 that extends proximally through the sheath 140. The first control wire 180 may be connected to the basket 130 through an elastic wire 160. The elastic wire 160 may be a length of wire at the distal end of the first control wire 180 that connects the basket 130 to the first control wire 180. The elastic wire 160 may be configured to stretch elastically and allow the basket 130 to extend distally with respect to the distal end of the first control wire 180 and invert or rotate about an axis (12, 14, etc.) perpendicular to a longitudinal axis of the sheath 140. In general, elastic wire 160 may have any elasticity. The elasticity of elastic wire 160 may be such that it may extend elastically to a length greater than its original upon application of a force and return to its original length upon removal of the force. In some embodiments, the elasticity and/or flexibility of elastic wire 160 may be more than that of the first control wire 180. In some embodiments, the elastic wire 160 may be more elastic and/or flexible than the first control wire 180 and the legs 136 of basket 130. The first control wire 180 and elastic wire 160 may be made of the same or different materials. If they are made of the same material, the dimensions/configuration of the wires may be such that the elastic wire 160 is more elastic and/or flexible than the first control wire 180 (for example, thinner, smaller cross-sectional area, etc.). If they are made of different materials, the elastic wire 160 may be made of a material that is more elastic and/or flexible than the first control wire 180 (for example, lower modulus of elasticity). In some embodiments, elastic wire 160 may be a compliant coiled spring or a length of coiled wire that readily deflects elastically upon the application of force. The distal end of the first control wire 180 may be joined to the proximal end of the elastic wire 160 by any method (for example, crimping, welding, adhesives, typing, hypo-tubes, etc.).

Device 100 may also include an engaging member 150 (or a forcing member) coupled to the basket 130. In some embodiments, engaging member 150 may include a substantially U-shaped strip (e.g., a flat strip) of an elastic material having a first end 152 and a second end 154. In its normal or unstressed state (that is, when a force is not applied to it), the engaging member 150 may have a substantially U-shaped configuration (as illustrated in FIG. 2). The engaging member 150 may be configured to elastically deform to a substantially linear configuration upon the application of a force and substantially return to its U-shaped configuration when the force is removed. The material of engaging member 150 may be the same as, or may be different from, the material of the basket 130. In some embodiments, engaging member 150 may be made of an elastic, super elastic, a spring material, or a shape-memory alloy.

The first end 152 of the engaging member 150 may be connected to the proximal end 132 of the basket 130 and the distal end of the elastic wire 160. As illustrated in FIG. 2, in some embodiments, the engaging member 150 may be connected to the proximal end 132 of the basket 130 such that a first portion 156 of the engaging member 150 at its first end 152 extends towards the distal end 134 of the basket 130. The engaging member 150 may then curve such that a second portion 158 proximate its second end 154 extends towards the sheath 140. The second end 154 of the engaging member 150 may be connected to a second control wire 170 that extends proximally through the sheath 140 alongside the first control wire 180. In embodiments where the sheath 140 includes a plurality of lumens, each of the first control wire 180 and the second control wire 170 may extend through separate lumens of sheath 140. The second control wire 170 may be similar to first control wire 180 and may be formed from similar materials (stainless steel, nitinol, spring steel, hybrid of a polymer and a metal, etc.). When no external force is applied on the engaging member 150, the engaging member 150 may remain in its U-shaped unstressed state (as illustrated in FIG. 2), and the basket 130 may be in its normal configuration. In this configuration, the distal portion 148 of the basket 130 may be positioned distal to the proximal portion 146 of the basket 130.

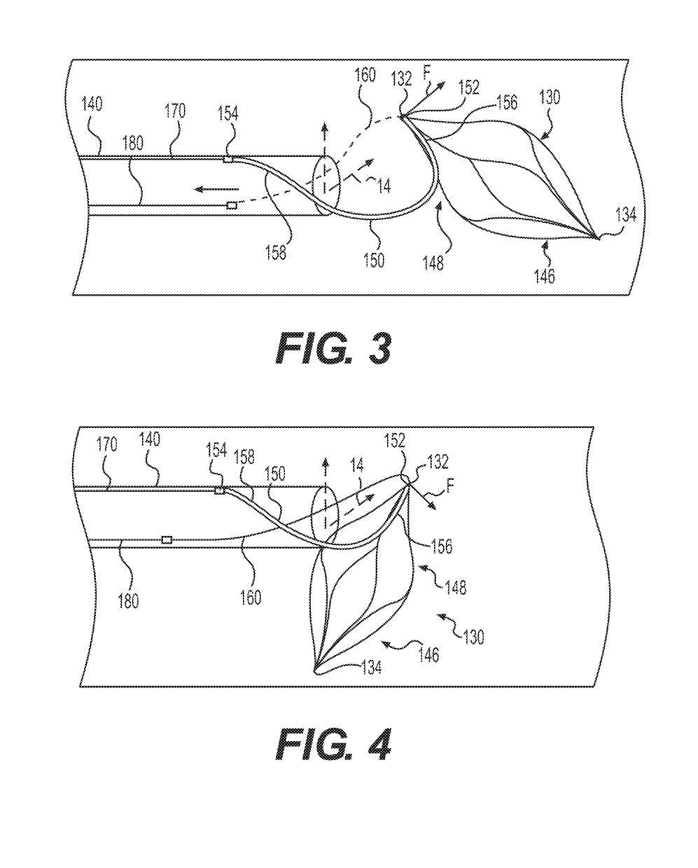

When the second control wire 170 is pulled proximally, the second end 154 of the engaging member 150 may be pulled into the sheath 140 thereby applying a force on the engaging member 150. FIG. 3 is a schematic illustration of the second end 154 of the control wire 150 being pulled into the sheath 140. As the U-shaped engaging member 150 gets pulled into the sheath 140, the inner walls of the sheath 140 may apply a reaction force on the engaging member 150 causing it to straighten. As the engaging member 150 straightens, a component of this reaction force, force F, may act at the first end 152 causing the first portion 156 of the engaging member 150, and the basket 130, to rotate (in a clockwise direction) about an axis 14 perpendicular to a longitudinal axis 10 the sheath 140. In some embodiments, the reaction force F may act in a direction normal to the first portion 156 of the engaging member 150. The magnitude of the force F may depend on, among others, the stiffness of the sheath 140 and the stiffness of the engaging member 150. Increased stiffness of the sheath 140 and/or the engaging member 150 may increase the magnitude of the force F, and decreased stiffness of the sheath 140 and/or the engaging member 150 may decrease the force F.

As the engaging member 150 continues to be pulled into the sheath 140, the first portion 156 of the engaging member 150 continues to rotate about axis 14, causing the basket 130 to invert (or flip) by rotating about axis 14. FIG. 4 is a schematic illustration of the inverted basket 130. In the inverted configuration, the distal portion 148 of the basket 130 may be positioned proximal to the proximal portion 146 of the basket 130. As the basket 130 inverts, the rotating first end 132 of the basket 130 may pull the first control wire 180 and the elastic wire 160 distally to allow the basket 130 to invert. In some embodiments, the elasticity of the elastic wire 160 may allow elastic wire 160 to extend by an amount necessary for the basket 130 to invert. In some embodiments, as the engaging member 150 is pulled in a proximal direction into the sheath 140, the first control wire 180 may be pushed in a distal direction to extend a length of the elastic wire 160 out of the sheath 140 to allow the basket 130 to invert.

When the basket 130 is in its inverted configuration, a stone captured in the basket 130 may escape from the basket 130 through the gap between its proximal legs 136a. In some embodiments, moving the sheath 140 along with the inverted basket 130 in the proximal direction, allows a captured stone in the basket 130 to be released back into the body lumen.

Pushing the second control wire 170 in a distal direction may extend the straightened engaging member 150 out of the sheath 140, thereby allowing the engaging member 150 to transform back to its unstressed U-shaped configuration, and the basket 130 back to its normal configuration (FIG. 2). As portions of the engaging member 150 extend from the sheath 140, the first portion 156 of the engaging member 150, and the basket 130, may rotate about axis 14 (in a counter clockwise direction) back to its normal configuration.

Engaging member 150 may have any suitable size and/or cross-sectional shape (round, rectangular, etc.). In general, the size and/or shape of engaging member 150 may be such that its memory force (force required to transform the engaging member 150 between its stressed and unstressed configurations) is high enough to invert the basket 130 and low enough to be applied through the second control wire 170. In some embodiments, engaging member 150 may be a wire made of a shape-memory material. The thickness and/or shape of the wire may be adapted to provide the desired memory force. Although the unstressed configuration of engaging member 150 is described as being U-shaped, this is only exemplary. In general, engaging member 150 may have any suitable shape (e.g., S-shaped) in its unstressed configuration.

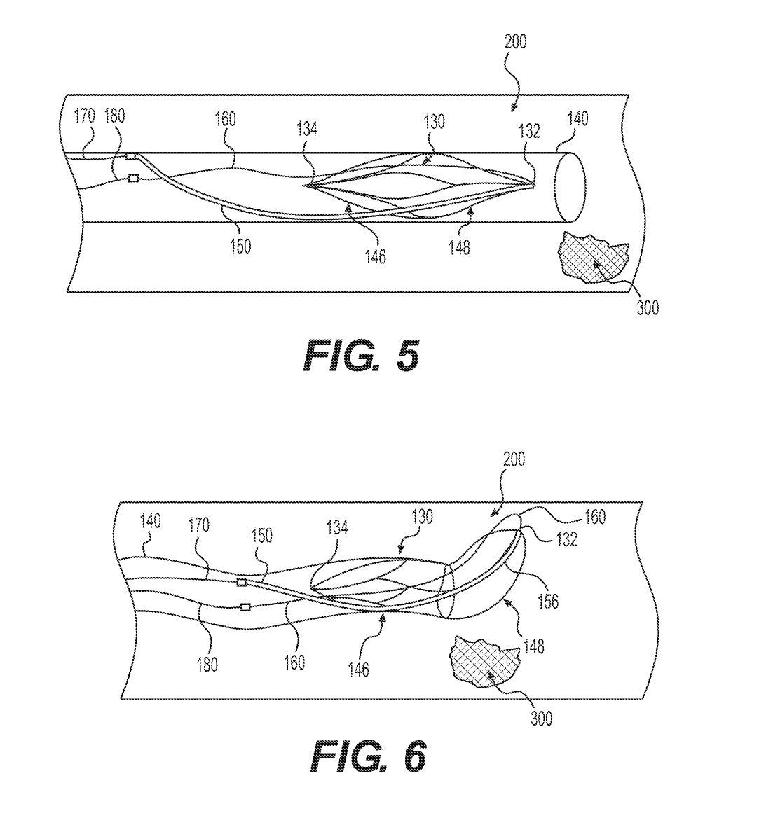

An exemplary method of using a medical device 100 of the current disclosure will be described below with reference to FIGS. 5-8. As illustrated in FIG. 5, the sheath 140, with the basket 130 loaded therein, may be introduced into a body lumen 200 of a patient through a natural anatomic opening or through a puncture. Within the sheath 140, the basket 130 may be positioned both in a radially compressed and an inverted configuration, with the engaging member 150 constrained in its stressed elongated configuration. The internal walls of the sheath 140 may apply the constraining force necessary to keep the engaging member 150 in the stressed configuration, and the basket 130 in its radially compressed configuration. In some embodiments, an external tension applied to second control wire 170 may assist in provide the necessary constraining force for the engaging member 150. With the distal end of the sheath 140 positioned adjacent to a stone 300 (or another material that is desired to be removed), the basket 130 may be deployed from the sheath 140 by pushing the first and second control wires 180, 170 in a distal direction.

FIG. 6 illustrates the distal portion 148 of the basket 130 and the first portion 156 of the engaging member 150 emerging from the sheath 140. As the basket 130 emerges from the sheath 140, it radially expands. As the engaging member 150 emerges from of the sheath 140, it curves to its stress free state, thereby rotating the first portion 156 of the engaging member 150, and the expanding inverted basket 130, in a counter clockwise direction. When the basket 130 and the engaging member 150 completely extends out of the sheath 140, the engaging member 150 transforms to its U-shaped unstressed configuration, and the basket transforms to its normal configuration (as illustrated in FIG. 2). After the basket transforms, in some embodiments, the first control wire 170 may be pulled in a proximal direction to remove any slack in the elastic wire 160.

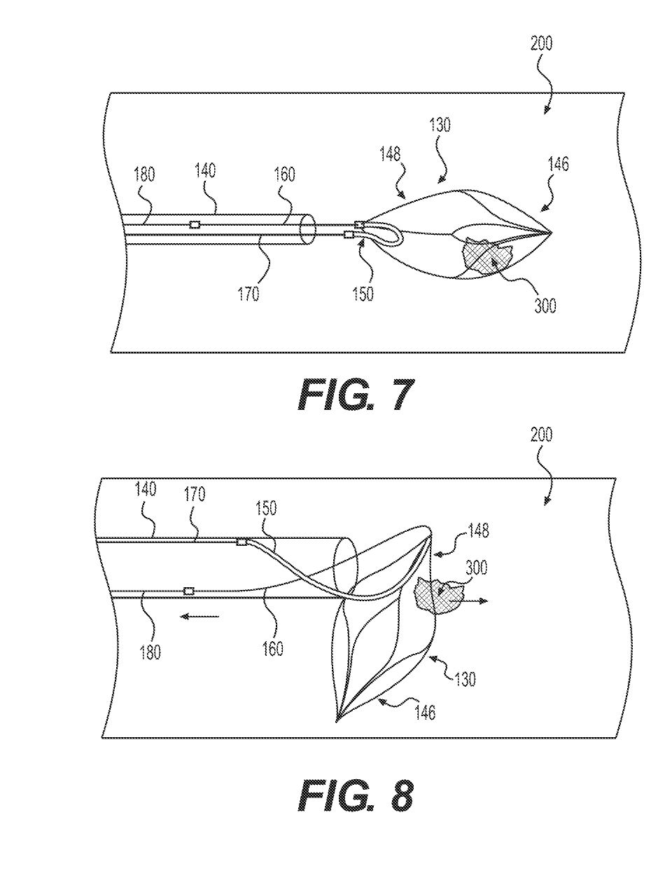

In the normal configuration, the basket 130 may be configured to capture and retrieve the stone 300. To capture the stone 300, the user may manipulate the basket 130 to trap the stone therein. In some embodiments, the user may pull the expanded basket in a distal direction over the stone 300 to sweep the stone 300 into the basket 130 through the gap between its proximal legs 136a. FIG. 7 illustrates the basket 130 in its normal configuration with the stone captured therein. The basket 130 with the stone 300 therein may then be removed from the body lumen 200. In some embodiments, the user may partially withdraw the basket 130 into the sheath 140 to tightly enclose the stone 300 in the partially withdrawn basket before removing the basket 130 from the body lumen 200.

If, however, the stone 300 is too large to be removed intact from the body lumen 200 after it has been captured within the basket 130, the user may invert the basket 130 to release the stone 300 back into the body lumen 200. To invert the basket 130, as discussed with reference to FIGS. 7 and 8, the user may pull the second control wire 170 in a distal direction to retract the engaging member 150 into the sheath 140. As the engaging member 150 is pulled into the sheath 140, it straightens and inverts the basket 130 with the stone therein. FIG. 8 illustrates an inverted basket with the stone therein. In the inverted configuration, the proximal portion 148 of the basket 130 is positioned distal to the distal portion 146. With the basket 130 in the inverted configuration, the basket 130 may be moved in the proximal direction to release the stone 300 from the basket 130 through the gap between its proximal legs 136a. Once the stone 300 is released from the basket 130, the user may push the second control wire 170 in a distal direction to transform the engaging member 150 back to its unstressed configuration and the basket 130 to its normal configuration (FIG. 7). The basket 130 may then be refracted into the sheath 140, and removed from the body lumen 200.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

We claim:

1. A medical retrieval device, comprising:
an elongate sheath;
a first and a second control wire extending through the sheath;
a basket coupled to a distal end of the first control wire; and
an engaging member coupled to a distal end of the second control wire and the basket, the engaging member being configured to transform from a constrained configuration within the sheath to an unconstrained configuration outside to the sheath, wherein transforming the engaging member from the unconstrained configuration to the constrained configuration rotates the basket about an axis perpendicular to a longitudinal axis of the sheath.

2. The device of claim 1, wherein the basket extends from a proximal end to a distal end, wherein a first end of the engaging member is connected to the proximal end of the basket and an opposite second end of the engaging member is connected to the distal end of the second control wire.

3. The device of claim 1, wherein in the unconstrained configuration, the engaging member is substantially U-shaped, and in the constrained configuration, the engaging member has an elongated shape.

4. The device of claim 1, further including an elastic wire connecting the distal end of the first control wire and the basket, wherein the elastic wire is more elastic than the first control wire.

5. The device of claim 4, wherein the elastic wire is a coiled wire.

6. The device of claim 1, wherein pulling the engaging member into the sheath transforms the engaging member to the constrained configuration and rotates the basket about the axis to an inverted configuration.

7. The device of claim 6, wherein the basket has a proximal portion and a distal portion, and wherein, in the inverted configuration, the proximal portion is distal to the distal portion.

8. The device of claim 1, wherein the basket and the engaging member include a shape-memory material or a spring material.

9. The device of claim 1, wherein the basket is configured to extend from within the sheath to outside the sheath, and wherein extending the basket from within the sheath to outside the sheath rotates the basket about the axis.

10. A medical retrieval device, comprising:
a sheath having a lumen;
a basket having a proximal end and a distal end, the basket configured to transition between a first configured within the lumen to a second configuration outside the lumen, wherein in the second configuration, the proximal end of the basket is positioned proximal to the distal end, and in the first configuration, the proximal end of the basket is positioned distal to the distal end; and
an engaging member coupled to the basket and configured to rotate the basket about an axis perpendicular to a longitudinal axis of the sheath as the basket emerges from within the lumen.

11. The device of claim 10, further including a first control wire and a second control wire extending through the sheath, wherein, a distal end of the first control wire is coupled to the proximal end of the basket, and a distal end of the second control wire is connected to a second end of the engaging member.

12. The device of claim 11, wherein a first end of the engaging member opposite the second end is connected to the proximal end of the basket.

13. The device of claim 11, further including an elastic wire connecting the distal end of the first control wire to the proximal end of the basket.

14. The device of claim 10, wherein retracting the engaging member from outside the lumen into the lumen is configured to rotate the basket about the axis.

15. A method of using a medical retrieval device to manipulate an object within a body of a patient, comprising:
directing a distal end of an elongate hollow sheath into the body, the sheath including a basket in a first configuration loaded therein, wherein in the first configuration, a proximal end of the basket is positioned distal to a distal end of the basket;
extending the basket from within the sheath to rotate the basket from the first configuration to a second configuration outside the sheath, wherein in the second configuration, the proximal end of the basket is positioned proximal to the distal end; and
capturing the object within the basket.

16. The method of claim 15, wherein extending the basket includes extending an engaging member out of the sheath, wherein the engaging member is coupled to the basket and is configured to transform from an elongated shape within the sheath to a curved shape outside the sheath.

17. The method of claim 16, further including retracting the engaging member into the sheath to rotate the basket about an axis perpendicular to a longitudinal axis of the sheath after capturing the object within the basket.

18. The method of claim 17, further including releasing the object from within the basket.

19. The method of claim 15, further including inverting the basket after capturing the object to release the object from the basket.

20. The method of claim 15, wherein extending the basket from within the sheath transforms the basket from a compressed configuration within the sheath to an expanded configuration outside the sheath.
